(19) United States
(12) Patent Application Publication
(10) Pub. No.: US 2010/0086587 A1
(43) Pub. Date: Apr. 8, 2010

(54) HARD SHELL CAPSULE FORMULATIONS

(76) Inventors: Victor Morrison Young, Edinburgh (GB); Stephen Brown, Essex (GB)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/518,989

(22) PCT Filed: Dec. 17, 2007
(86) PCT No.: PCT/GB07/04854

§ 371 Date: Dec. 2, 2009

(30) Foreign Application Priority Data
Dec. 15, 2006 (GB) 0625180.5

(51) Int. Cl. A61K 9/66

(52) U.S. Cl. 424/455

(57) ABSTRACT

Hard shell capsules filled with a normally hygroscopic formulation and a method for producing the same is described. The hard shell capsules are filled with a normally hygroscopic formulation including, for example, an excipient, and optionally an active compound. The water content of the formulation and the hard shell capsule are in equilibration, and the formulation is hydrated by an amount selected to control equilibrium between said formulation and the hard shell capsule, and to provide a structurally stable hard shell capsule. The active compound may be a protein or peptide.

### TECHNICAL FIELD

This invention relates to hard shell capsule formulations, and in particular to hard shell capsules filled with a normally hygroscopic formulation and a method for producing the same.

The present invention is concerned with the liquid filling of hard shell capsules where liquid filling is described as the filling of a material that has the properties of a liquid at the point of filling. This means that the material to be filled may range from a room temperature liquid through to high temperature thermosoftening material (melting) or thixotropic gels, suspensions or any combination of these.

### BACKGROUND ART

Hard shell and soft shell capsules are well known in the field of pharmaceuticals and nutraceuticals and the like. Both types of capsule have their pros and cons and choosing the type of capsule to use depends on many factors including the nature of the agents to be encapsulated. In this regard, a problem associated with hard shell capsules is that they cannot be used to encapsulate hygroscopic materials including actives and excipients such as some polyethylene glycols, sugars and lactams.

Polyethylene glycols (PEGs) are condensation polymers formed from ethylene oxide and water with molecular weights varying from 200 to over 8000 units. The low molecular weight materials (<700) are hygroscopic liquids while those of 1000 units or greater are waxy solids with little hygroscopicity.

PEGs are stable compounds that have been widely used in pharmaceutical formulations as they are good solvents, water soluble and, in particular, enhance compound bioavailability. They have been used in parenteral, topical, oral, ophthalmic and rectal formulations and are generally regarded as non-toxic and non-irritant materials. They are included in the FDA Inactive Ingredient Guide for dental preparations, IM and IV injections, ophthalmic preparations, oral capsules, solutions, syrups, tablets plus rectal, topical and vaginal preparations. Liquid PEGs may be absorbed from oral dosage forms while higher (solid) molecular weight materials are not significantly absorbed. The World Health Organisation (WHO) has set an estimated acceptable daily intake at up to 10 mg/kg for PEGs.

Solid (i.e. high molecular weight) PEGs have been used extensively in dosage forms, including hard shell capsules, as they are neither hygroscopic nor incompatible with the shell materials of hard capsules. These solid PEGs are generally melted and then pumped into hard shell capsules during manufacture. Although these solid PEGs are compatible with hard shell capsules, solid PEGs do not have the same advantageous properties as liquid PEGs. In particular, they do not have the same capability of markedly increasing the bioavailability of active compounds that liquid PEGs have.

As liquid PEGs are highly hygroscopic, they destroy hard shells manufactured from materials such as gelatin, hydroxypropyl methylcellulose (HPMC), and pullulan when used at anything other than very low concentration (<1-2% w/w). It is known in the art that the presence of anything other than trace amounts of liquid PEGs in hard shell capsules leads to embrittlement, or splitting of the capsule. Such low concentrations are generally too small to provide any of the desirable effects of liquid PEGs such as a marked improvement of the bioavailability of the compound contained in the capsule formulation.

Therefore although liquid PEGs have been employed widely in formulations in soft gel capsules, they have not been used in hard shell capsules due to their destructive effect on the capsule shell. The same problematic effects are seen with other hygroscopic active materials, and inactive excipients including some sugars and some lactams.

It is particularly desirable to produce capsules that enable the administration of proteins and peptides. Pharmaceutical proteins and peptides are generally produced from mammalian or bacterial cell fermentation processes and the final protein or peptide is usually harvested and purified into a suitable aqueous buffer system. Proteins and peptides are generally most stable when they are in aqueous environments such as water or buffers (e.g., citrate, acetate, phosphate buffered saline, plasma). However, proteins and peptides intended for oral administration are often lyophilized or freeze-dried for stability reasons or for ease of processing into dry powder pharmaceutical dosage forms. This often results in structural and conformational changes which may not be reversible when the protein/peptides are reconstituted following oral administration. It would therefore be of benefit to be able to incorporate proteins and peptides in their preferred conformation in water or aqueous buffer solutions and in a suitable oral dosage form.

Accordingly, it is an object of the present invention to provide improved hard shell capsules. In particular it is an object of the invention to provide stable hard shell capsules filled with a normally hygroscopic formulation. It is a further object of this invention to provide a method for manufacturing improved hard shell capsules, and a method for filling hard shell capsules with normally hygroscopic formulations.

### DISCLOSURE OF INVENTION

According to the first aspect of the present invention there is provided a method of encapsulating a formulation, the method comprising the steps of providing a hard shell capsule, and a normally hygroscopic formulation to be filled into said capsule, hydrating said formulation by a selected amount to control anticipated equilibrium between said formulation and the hard shell capsule, filling said hydrated formulation into said hard shell capsule to provide a structurally stable hard shell capsule.

The hygroscopic component is a compound (active or excipient) which would normally be incompatible with hard shell capsules. The formulation is hydrated through the addition of a selected amount of water or aqueous solution prior to the filling process. The capsule may then be sealed. The water content of the capsule fill material is then in equilibrium with the water content of the capsule shell.

Sufficient aqueous medium (buffer, water etc) can be used during the formulation phase such that the amount of water present in the formulation (e.g., in any excipients and the active compound) will be in equilibrium with the water content of the hard shell capsule, and such that a structurally stable hard shell capsule is produced.

The hydration may take place by adding the water or aqueous solution to the hygroscopic excipient followed by the addition of the active compound before filling the final formulation into the hard shell capsule. Alternatively hydration may take place by adding the water or aqueous solution to the hygroscopic excipient to which the active had already been added. In a further alternative hydration may take place by combining the water or aqueous solution and the active to create a solution or suspension which may then be added to the hygroscopic excipient. For example, one or both of the excipient and the active compound can be hydrated before they are incorporated into the final formulation before addition to the hard shell capsule.

The hard shell capsule may comprise a material chosen from the list comprising gelatin, hydroxypropyl methylcellulose, pullalan, starch, polyvinyl alcohol and suitable derivatives thereof.

Preferably the formulation comprises at least one hygroscopic excipient. The hygroscopic excipient may be chosen from the list comprising hygroscopic polyethylene glycols, hygroscopic sugars, and hygroscopic lactams, and suitable derivatives thereof.

When the hygroscopic excipient is polyethylene glycol, the polyethylene glycol may be hydrated to between about 20% w/w and about 32% w/w water with respect the polyethylene glycol content. The water content may be between about 24% w/w and about 28% w/w water with respect to the polyethylene glycol content.

When the hygroscopic excipient is a sugar, the sugar may be hydrated to between about 11% w/w and about 16% w/w water. The water content may be between about 13% w/w and about 14% w/w water with respect to the sugar content.

When the hygroscopic excipient is a lactam, the lactam may be hydrated to between about 6.5% w/w and about 10% w/w water. The water content may be between about 8.5% w/w and about 9.5% w/w water with respect to the lactam content.

Optionally the formulation comprises at least one active compound. The active compound may be a protein or peptide, or suitable derivative thereof.

According to a second aspect of the invention there is provided a capsule manufactured by the method substantially as described herein.

According to a third aspect of the invention there is provided a capsule comprising a hard shell filled with a hydrated, normally hygroscopic, formulation the water content of the hydrated formulation being an amount selected to control the equilibrium between said formulation and the hard shell and to provide a structurally stable capsule.

The hard shell may comprise a material chosen from the list comprising gelatin, hydroxypropyl methylcellulose, pullalan, starch, polyvinyl alcohol and suitable derivatives thereof.

Preferably the formulation comprises at least one hygroscopic excipient. The hygroscopic excipient may be chosen from the list comprising hygroscopic polyethylene glycols, hygroscopic sugars, and hygroscopic lacatams, and suitable derivatives thereof.

When the hygroscopic excipient is polyethylene glycol, the polyethylene glycol may comprise between about 20% w/w and about 32% w/w water. The water content may be between about 24% w/w and about 28% w/w water with respect to the polyethylene glycol content.

When the hygroscopic excipient is a sugar, the sugar may comprise between about 11% w/w and about 16% w/w water. The water content may be between about 13% w/w and about 14% w/w water with respect to the sugar content.

When the hygroscopic excipient is a lactam, the lactam may comprise between about 6.5% w/w and about 10% w/w water. The water content may be between about 8.5% w/w and about 9.5% w/w water with respect to the lactam content.

Optionally the formulation comprises at least one active compound. The active compound may be a protein or peptide, or suitable derivative thereof.

### MODES FOR CARRYING OUT THE INVENTION

A general technique for preparing hard shell capsules of the present invention is as follows. An excipient and water are mixed to form a clear solution. Where an active compound is included, it can be added to either the water or the hygroscopic excipient before these are combined or may be added to the formulation after the water and hygroscopic excipient have been combined. Hard shell capsules are filled with the so-formed solution or suspension and are sealed by banding. Capsules are then packaged into appropriate secondary packaging for clinical use.

During evaluation of formulations capsules were leak tested and satisfactory capsules were packed into sealed glass bottles. The sealed glass bottles were placed on stability at 40° C. for accelerated stress testing. Samples were removed and examined periodically over a three month period and were examined for leakage and embrittlement or softening of the capsules.

Capsules which contain too much water, or aqueous solution or buffer, will soften due to increased moisture uptake by the capsule shell, and hence be unacceptable. Capsules which contain insufficient water, or aqueous solution or buffer, will become embrittled and will be susceptible to cracking and leaking due to moisture transfer from the capsule shell to the formulation. Capsules were manually compressed and flexed to assess brittleness or softening in comparison to unfilled control capsules. The range of water content (with respect to hygroscopic excipient) in the formulations that produced structurally stable capsules (i.e. not embrittled or softened) was thus determined.

As alluded to previously, liquid PEGs having average molecular weights of up to around 1000 are hygroscopic. Grades of liquid PEG falling within this range that are commonly used as excipients include PEG 200, PEG 300, PEG 400 and PEG 600 (the number denoting the average molecular weight). PEG 400 was used as a mid range typical hygroscopic PEG in trials, however these results are typical of all liquid PEGs. Liquid PEGs are understood to be PEGs with an average molecular weight below 1000, and that are liquid at room temperature.

Several PEG 400/water mixes were prepared containing 5, 10, 15, 20, 22, 24, 25, 26, 28, 30, 32, 34, 35 and 40% water. Hard shell capsules were then filled with the mixes. Note that this concentration data is a summary of data obtained over a series of experiments.

Samples of all capsules were placed on a stability trial for three months. Capsules containing PEG 400 with 20% w/w to 32% w/w water content were found to be stable after three months; that is they retained their structural integrity. In particular, capsules containing 24% w/w to 28% w/w water were found to be stable after three months.

As mentioned above, when the hygroscopic excipient is polyethylene glycol, the polyethylene glycol may be hydrated to between about 24% w/w and about 28% w/w water. However, the optimal value will be dependent on polyethylene glycol MW used, and on the type of hard shell material used, and it has been found that stable capsules can be obtained when the liquid PEG contains 20% w/w to 32% w/w. water.

2-Pyrrolidone is a hygroscopic pharmaceutical lactam excipient which was similarly examined to determine how much water is required to produce a stable gelatin capsule. Testing was carried out as described above using capsules prepared with 8, 10, 12, 14, 15, 16, 17, 18, 20 25 and 30% water concentration in 2-pyrrolidone. Following the initial trials, further experiments were carried out using capsules containing 2-pyrrolidone with a water content of 6.5, 7.0, 7.5, 8.0, 8.5, 9.0 and 9.5% water. Capsules with a 2-pyrrolidone water content of between 6.5% w/w and 10% w/w, and more particularly between 8.5% w/w and 9.5% w/w, were found to be stable.

As noted above, when the hygroscopic excipient is a lactam, the lactam may be hydrated to between about 8.5% and about 9.5% water. However, this range of values is dependent on the lactam used, and the type of hard shell material used, and it has been found that stable capsules can be obtained when the lactam contains 6.5% w/w to 10% w/w water.

An alternative method for evaluating the level of water required to provide a stable hygroscopic formulation involves the preparation of capsules containing different water contents and then quantifying moisture loss or gain when exposed to a 50% RH environment (middle of acceptable RH range for capsules 35% RH to 65% RH). Maltitol, a sugar which is also a hygroscopic excipient, was dissolved in water to produce several aqueous maltitol solutions of concentration 16, 18, 20, 22, 24, 26, 28, 30, 32, 34 and 36% water in maltitol. Both gelatin and HPMC capsules were filled with the various concentrations of solution and the capsules were sealed by banding. The capsules were then exposed to a 50% R.H environment and allowed the capsules to re-equilibrate.

Capsules were weighed at intervals and it was noted that they progressively reduced in weight as water was lost from the formulation. Weight loss (and water loss) ceased when the capsules shell content was in equilibrium with the capsule shell and with the 50% R.H. environment. The equilibrium water content in maltitol that produced stables capsules was approximately 14% water content for gelatin capsules and approximately 13% water content for HPMC capsules.

As noted above, when the hygroscopic excipient is a sugar, the sugar may be hydrated to between about 13% and about 14% water. It will be appreciated that this range of values is variable depending on the type of sugar used, and the type of hard shell material used, and it has been found that stable capsules can be obtained when the sugar contains 11% w/w to 16% w/w water.

The formulations described above can comprise an active compound such as a pharmaceutical or nutraceutical, or a protein or a peptide, or suitable derivative thereof.

The method as described is useful for encapsulating a formulation. The method involves providing a hard shell capsule, filling the hard shell capsule with at least one normally hygroscopic excipient, hydrating the formulation, filling the formulation into hard shell capsules and sealing the hard shell capsules.

The capsule produced comprises a hard shell filled with a hydrated, normally hygroscopic, formulation wherein the water content of the formulation is in equilibrium with the water content of the hard shell.

Sufficient aqueous medium (buffer, water etc) can be provided to facilitate formulation. The amount of water present in the formulation (e.g., in the excipient and the active compound) may be in equilibrium with the water content of the hard shell capsule.

The hydration would normally take place before the addition of the hygroscopic excipient and the active compound formulation to the hard shell capsule. For example, one or both of the excipient and the active compound can be hydrated before they are combined and before addition to the hard shell capsule.

The hard shell capsule may be made from a material chosen from the list comprising gelatin, hydroxypropyl methylcellulose, pullalan, starch, polyvinyl alcohol and suitable derivatives thereof, and other suitable hard shell materials known in the art.

As noted above the formulation comprises at least one hygroscopic excipient which may be chosen from the list comprising hygroscopic polyethylene glycols, hygroscopic sugars, and hygroscopic lacatams, and suitable derivatives thereof, and other suitable hygroscopic excipient materials known in the art.

Hard shell capsules such as those based on gelatin or HPMC remain flexible and usable as long as their water content remains within particular limits, being approximately 13%-16% w/w for gelatin and 5%-10% w/w for HPMC. These figures are provided for illustration and accurate figure can be obtained from individual capsule manufacturers. Note that hard shell capsules can also be based on pullalan or other suitable hard shell capsule materials known to the art e.g. starch or polyvinyl alcohol combinations.

In general, hard shell capsules remain stable and usable as long as their water content remains within certain values which are particular to the shell material. Until now it has been thought that it is impossible to maintain hard shell capsule water content within these limits in the presence of liquid PEGs and other hygroscopic excipients including propylene glycol, lactams and sugars.

The general principle remains that it is possible to manufacture a stable hard shell filled capsule containing significant quantities of liquid PEG and other hygroscopic excipients by the addition of a particular percentage of water. This achieves a balance between the formulated contents and the capsule shell such that the contents of the capsule and the shell material do not make a net gain or loss of water from each other when in intimate contact.

Accordingly there is provided the availability to use hygroscopic excipients such as liquid PEGs, at significant quantity, in hard shell encapsulation by using added water to balance the hygroscopicity of the formulated materials (principally the liquid PEG) with that of the shell wall, resulting in a stable dosage unit.

### EXAMPLES

### Example 1

A water soluble protein (HF1020-EtxB), which is stable in an aqueous environment, was provided as a concentrate in an aqueous buffer (phosphate buffer solution (PBS)), which was diluted to the required concentration with water. The diluted protein/buffer solution was then mixed with PEG 400 excipient, the so-formed formulation containing 28% water. A hard shell gelatine capsule was then filled with the formulation and the capsule was closed and banded. After stability testing, the capsule was found to remain stable without softening or cracking after three months stability storage, and the formulation retained its protein concentration and activity. Thus there is provided a viable route for the formulation of proteins (and peptides) in stable aqueous media for administration in capsule form.

### Example 2

A water soluble protein, which is stable in an aqueous environment, was provided as a concentrate in an aqueous buffer (phosphate buffer solution (PBS)), which was diluted to the required concentration with water. The diluted protein/buffer solution was then mixed with PEG 400 excipient, the so-formed formulation containing 24% water. A hard shell gelatine capsule was then filled with the formulation and the capsule was closed and banded. After exposure to stability testing, the capsule was found to remain stable.

### Example 3

The excipient 2-pyrrolidone was mixed with water to form a clear solution with 8.5% water content. A hard shell gelatin capsule was filled with the solution, and the capsule was closed and banded. After exposure to stability testing, the capsule was found to remain stable without softening or cracking after three months stability storage.

### Example 4

The excipient 2-pyrrolidone was mixed with water to form a clear solution with 9.5% water content. A hard shell gelatin capsule was filled with the solution, and the capsule was closed and banded. After exposure to stability testing, the capsule was found to remain stable without softening or cracking after three months stability storage.

### Example 5

The excipient maltitol, was mixed with water to form a solution with 14% water content. A hard shell gelatin capsule was filled with the solution, and the capsule was closed and banded. After exposure to stability testing, the capsule was found to remain stable without softening or cracking after three months stability storage.

### Example 6

The excipient maltitol, was mixed with water to form a solution with 13% water content. A hard shell HPMC capsule was filled with the solution, and the capsule was closed and banded. After exposure to stability testing, the capsule was found to remain stable without softening or cracking after three months stability storage.

It is an unusual finding that significant quantities of water or buffer can be incorporated into hard shell capsules, as this was previously thought to produce only unstable capsules.

Furthermore, the capsules of the present invention enable proteins or peptides (prepared in an aqueous environment) to be incorporated into water or buffer systems and subsequently formulated into hard capsule formulations with PEG or other hygroscopic vehicles. This is advantageous over the more usual route of lyophilisation/freeze drying of proteins or peptides because it facilitates delivery of the protein or peptides in their active conformation in their preferred environmental conditions. This increases the likelihood of the protein being delivered in a stable and active conformation.

### INDUSTRIAL APPLICABILITY

The invention as described herein has applications in the field of pharmaceutical and nutraceutical delivery. In addition, it has a general application in the field of encapsulation and in particular hard shell encapsulation.

Improvements and modifications may be incorporated herein without, deviating from the scope of the invention.

What is claimed is:

1. A method of encapsulating a formulation, the method comprising the steps of providing a hard shell capsule, and a normally hygroscopic formulation to be filled into said capsule, hydrating said formulation by a selected amount to control anticipated equilibrium between said formulation and the hard shell capsule, filling said hydrated formulation into said hard shell capsule to provide a structurally stable hard shell capsule.

2. A method as described in claim 1 wherein the hard shell capsule comprises a material chosen from the list comprising gelatin, hydroxypropyl methylcellulose, pullalan, starch, polyvinyl alcohol and suitable derivatives thereof.

3. A method as described in claim 1 wherein the formulation comprises at least one hygroscopic excipient.

4. A method as described in claim 3 wherein the hygroscopic excipient is chosen from the list comprising hygroscopic polyethylene glycols, hygroscopic sugars, and hygroscopic lacatams, and suitable derivatives thereof.

5. A method as described in claim 4 wherein the hygroscopic excipient is polyethylene glycol and wherein the polyethylene glycol is hydrated to between about 20% w/w and about 32% w/w water.

6. A method as described in claim 5 wherein the polyethylene glycol is hydrated to between about 24% w/w and about 28% w/w water.

7. A method as described in claim 4 wherein the hygroscopic excipient is a sugar and wherein the sugar is hydrated to between about 11% w/w and about 16% w/w water.

8. A method as described in claim 7 wherein the sugar is hydrated to between about 13% w/w and about 14% w/w water.

9. A method as described in claim 4 wherein the hygroscopic excipient is a lactam and wherein the lactam is hydrated to between about 6.5% w/w and about 10% w/w water.

10. A method as described in claim 9 wherein the lactam is hydrated to between about 8.5% w/w and about 9.5% w/w water.

11. A method as described in claim 1 wherein the formulation comprises at least one active compound.

12. A method as described in claim 11 wherein the active compound is a protein or peptide, or suitable derivative thereof.

13. A capsule manufactured by the method substantially as described in claim 1.

14. A capsule comprising a hard shell filled with a hydrated, normally hygroscopic, formulation the water content of the hydrated formulation being an amount selected to control the equilibrium between said formulation and the hard shell and to provide a structurally stable capsule.

15. A capsule as described in claim 14 wherein the hard shell comprises a material chosen from the list comprising gelatin, hydroxypropyl methylcellulose, pullalan, starch, polyvinyl alcohol and suitable derivatives thereof.

16. A capsule as described in claim 14 wherein the formulation comprises at least one hygroscopic excipient.

17. A capsule as described in claim 14 wherein the hygroscopic excipient is chosen from the list comprising hygroscopic polyethylene glycols, hygroscopic sugars, and hygroscopic lacatams, and suitable derivatives thereof.

18. A capsule as described in claim 17 wherein the hygroscopic excipient is polyethylene glycol and wherein the polyethylene glycol comprises between about 20% w/w and about 32% w/w water.

19. A capsule as described in claim 18 wherein the polyethylene glycol comprises between about 24% w/w and about 28% w/w water.

20. A capsule as described in claim 17 wherein the hygroscopic excipient is a sugar and wherein the sugar comprises between about 11% w/w and about 16% w/w water.

21. A capsule as described in claim 20 wherein the sugar comprises between about 13% w/w and about 14% w/w water.

22. A capsule as described in claim 17 wherein the hygroscopic excipient is a lactam and wherein the lactam comprises between about 6.5% w/w and about 10% w/w water.

23. A capsule as described in claim 22 wherein the lactam comprises between about 8.5% w/w and about 9.5% w/w water.

24. A capsule as described in claim 14 wherein the formulation comprises at least one active compound.

25. A capsule as described in claim 24 wherein the active compound is a protein or peptide, or suitable derivative thereof.
